(19) United States
(12) Patent Application Publication
(10) Pub. No.: US 2023/0363378 A1
(43) Pub. Date: Nov. 16, 2023

(54) METHOD FOR CRYOPRESERVING A PLURALITY OF CELL CLUSTERS OF BIOLOGICAL CELLS

(71) Applicant: Fraunhofer-Gesellschaft zur Foerderung der angewandten Forschung e. V., Munchen (DE)

(72) Inventors: Heiko ZIMMERMANN, Sulzbach (DE); Ina MEISER, Sulzbach (DE); Frank STRACKE, Sulzbach (DE)

(21) Appl. No.: 18/029,354

(22) PCT Filed: Oct. 5, 2021
(86) PCT No.: PCT/EP2021/077425

§ 371 Date: Mar. 29, 2023

(30) Foreign Application Priority Data
Oct. 22, 2020 (DE) 10 2020 127 787.3

(51) Int. Cl. A01N 1/02

(52) U.S. Cl. CPC A01N 1/0284; A01N 1/0257

(57) ABSTRACT

A method for cryopreserving a plurality of cell clusters (1, 2) of biological cells includes the steps of fractionating the cell clusters (1, 2) into at least two fractions (4) in dependency on at least one property of the cell clusters (1, 2), collecting the fractions (4) in different containers (21), and cryopreserving the cell clusters (1, 2) of the at least two fractions (4), wherein specific pretreatment methods and/or freezing methods are used for each fraction. A cryopreservation apparatus (100), for cryopreserving a plurality of cell clusters (1, 2) of biological cells, having a fractionation device, is also described.

The invention relates to a method and a cryopreservation apparatus for cryopreserving a plurality of cell clusters of biological cells (also referred to as cell aggregates), for example of cellular tissue or organoids. The invention has applications for example in biomedicine and/or biotechnology.

In biotechnological/pharmacological research and in biomedicine, for example in transplant medicine, there is an interest in applications for cell clusters of a plurality of biological cells. In particular, cell clusters make it possible to emulate specific properties of functions of organs of a biological organism without forming a complete organ. Cell clusters comprise, for example, native biological tissue (cell-matrix clusters grown in vivo), spheroids (spherical clusters of cells) or organoids (cell-matrix clusters grown artificially in vitro). The formation of cell clusters may require culture times of days, weeks, or even months, with individual cell clusters developing at different rates. As a result, culturing methods typically produce inhomogeneous samples with cell clusters at different stages of maturity or development, in particular of different sizes.

The cryopreservation of biological materials, for example cells, cell components and/or groups of cells, is a generally known method by means of which biological materials are frozen while retaining vitality. The freezing takes place according to predetermined freezing protocols, typically using a cryoprotectant (CPA) which prevents or suppresses the formation of ice crystals during the freezing process. Process parameters (conditions of pretreatment and subsequent freezing) are selected in particular on the basis of the properties of the biological materials (see for example the review by M. A. Taylor et al. “New Approaches to Cryopreservation of Cells, Tissues and Organs” in “Transfus. Med. Hemother.” 46: 2019, 197-215).

For the effective cryopreservation of suspensions of individual cells with a high vitality yield, cell-type-specific process parameters, for example composition of auxiliary agents, duration of action and cooling rate, are optimized, which may require a large amount of effort and a considerable amount of experience. In the cryopreservation of cell clusters, the success of the preservation is even more acutely dependent on the selection of the process parameters, meaning that the effort in selecting optimized freezing parameters also grows.

While the cryopreservation of individual cells in suspension is seeing more widespread use particularly by virtue of slow freezing, inhomogeneous samples of expanded, three-dimensional cell clusters, for example tissues, spheroids or organoids, of different sizes have to date only been freezable in limited yields using slow freezing. For example, the required duration of action of auxiliary agents leading to a desired concentration inside the cell cluster increases fourfold with the diameter of the cluster. Since the above-mentioned inhomogeneity, in particular polydispersity, typically occurs in the case of customary (scalable) production methods for cell clusters, it should also be expected that, in the case of a preservation protocol which has been optimally selected in relation to the cell type and for a particular size, losses will occur due to the unsuitable durations of action and conditions for deviating sizes. In the conventional cryopreservation of a polydisperse sample of cell clusters, therefore, it has to date always been necessary to accept compromises in the selection of the freezing parameters.

It is also known to preserve relatively small cell clusters using vitrification (vitrification by ultra-rapid cooling). However, very stringent technical limits are imposed on vitrification in order to provide very high concentrations of auxiliary agents and to reach temperatures below the glass transition temperature ultra-rapidly at every point in the three-dimensional tissue cluster. This relates above all to the volume of the sample, which has an upper limit due to the limited thermal conductivity of aqueous media (λ = 0.56 W/Km, α = 0.14 mm²/s). However, the auxiliary agent concentrations and durations of action of the auxiliary agents are also limited by the cytotoxicity of said agents. In addition, the probability of damage due to thermal stress cracks increases with increasing sample size. Vitrification is therefore unsuitable for the routine preservation of samples having a plurality of cell clusters of different sizes.

In practice, said limitations on the selection of freezing parameters do not only occur when cryopreserving samples having cell clusters of different sizes. The inhomogeneity of a sample can result from various other properties of cell clusters prepared together, for example different shapes or elasticities.

The objective of the invention is that of providing an improved method and an improved cryopreservation apparatus for cryopreserving a plurality of cell clusters of biological cells, by means of which disadvantages of conventional techniques can be avoided. It is intended in particular to improve the cryopreservation of cell clusters in relation to the optimization of process parameters, yield, efficiency and/or usability for various cell types.

This objective is achieved by a method and a cryopreservation apparatus for cryopreserving a plurality of cell clusters of biological cells having the features of the independent claims. Advantageous embodiments and applications of the invention are given by the dependent claims.

According to a first general aspect of the invention, the above-mentioned objective is achieved by a method for cryopreserving a plurality of cell clusters of biological cells, comprising the steps of fractionating the cell clusters into at least two fractions on the basis of at least one predetermined property of the cell clusters, collecting the fractions in different containers, and cryopreserving the cell clusters in the at least two fractions, wherein specific pretreatment methods and/or freezing methods are used for each fraction.

According to a second general aspect of the invention, the above-mentioned objective is achieved by a cryopreservation apparatus, which is adapted for cryopreserving a plurality of cell clusters of biological cells, and comprises a fractionation device, which is adapted for fractionating the cell clusters into at least two fractions on the basis of at least one predetermined property of the cell clusters, a container device having at least two different containers, each of which is arranged for collecting one of the fractions, and a freezing device, which is adapted for cryopreserving the cell clusters in the at least two fractions, wherein the freezing device is adapted for applying specific pretreatment methods and/or freezing methods for each of the fractions. Preferably, the cryopreservation apparatus, or an embodiment thereof, is adapted to carry out the method for cryopreservation according to the first general aspect of the invention or an embodiment of the method.

According to the invention, the cell clusters are fractionated into at least two fractions on the basis of at least one predetermined property of the cell clusters. Typically, up to 5 or up to 10 fractions are formed. However, more fractions, for example up to 20 or more, can also be provided.

Advantageously, at least two homogeneous fractions are created by the fractionation of the cell clusters, for each of which fractions process parameters for the cryopreservation can be optimized. The inventors have found that optimal process parameters for cryopreservation are not only dependent on the cell type but also on properties of a cell cluster per se, for example the size of the cell cluster. The inventors have furthermore found that, in conventional methods, different sizes of cell clusters within an inhomogeneous fraction have the effect that, for each size, auxiliary agents and water are distributed differently within the cell cluster, thereby having different effects on the freezing, which is disadvantageous for the success and the yield of the cryopreservation. By creating, according to the invention, homogeneous fractions, the limitations of the conventional processing of inhomogeneous fractions are overcome.

The preparation of homogeneous fractions has a further advantage for applications of cell clusters, for example for research purposes or for an implant treatment, where there is an interest in fractions having cell clusters at the same, or similar, stages of maturity or development. Such fractions can be formed by similar cell clusters being obtained for a desired purpose using a culture method, being subjected to cryopreservation and being stored in the frozen state.

The term “cell cluster” denotes a contiguous, preferably three-dimensionally extending groups of living biological cells, like for example a tissue (in particular a tissue model), a spheroid or an organoid. The cell cluster may consist exclusively of cells or may also contain extracellular matrix substances in addition to the cells. Cell clusters are prepared for example by culturing biological cells and/or by extraction from an organism. The term “fraction” denotes a plurality of cell clusters in a liquid surrounding medium.

The fractionation comprises a separation (sorting, separation process) of cell clusters from an initially inhomogeneous sample into a predetermined number of fractions. The separation takes place such that each of the fractions contains cell clusters and the cell clusters in each of the fractions have at least one equal property. This means that the cell clusters in each of the fractions are identical in relation to the at least one property or have such minor differences that said differences do not have an effect on the cryopreservation, in particular the selection of optimal parameters for the pretreatment method and the freezing method. Each of the fractions is homogeneous in relation to the at least one property in question. The fractionation is in particular a separation method which preferably does not change the cell clusters. In particular, the cell clusters remain intact upon fractionation; in other words, the cell clusters are not broken up into pieces.

The fractionation device preferably comprises a separation device which is adapted to accommodate a composition of the cell clusters in a surrounding medium to separate the cell clusters into the different fractions and to dispense the fractions into the different containers.

According to the invention, the fractions are collected in different containers, i.e. the fractionation comprises separation into different containers. Each container generally comprises a receptacle for the fraction, which typically comprises the equal cell clusters and a liquid surrounding medium, for example a nutrient medium. The receptacles of the different containers are separated from one another.

Preferably, the fractions are collected in the containers in which the cryopreservation subsequently takes place. Advantageously, as a result, the method composed of fractionation and cryopreservation, and the structure of the cryopreservation apparatus, are simplified, and some undesired influences on the cell clusters are prevented after fractionation.

In the cryopreservation, the separated fractions obtained during the fractionation are frozen. Alternatively, before freezing, the fractions may be subjected to a change of the surrounding medium and/or an enrichment of the cell clusters in the surrounding medium.

The cryopreservation of the cell clusters comprises a pretreatment method and subsequently a freezing method. The pretreatment (or incubation) of the cell clusters comprises preparing the cell clusters for freezing, with for example the composition of the liquid surrounding medium being adjusted with auxiliary agents or CPA (the entirety of all additives used for improving the preservation result), the volume of the fraction being adjusted, the density of the cell clusters in the fraction being adjusted and/or other pretreatment parameters being adjusted and/or the cell clusters being changed, for example permeabilized, using physical and/or chemical methods. The pretreatment of the cell clusters preferably takes place at a temperature at which the surrounding medium is liquid, in particular at room temperature. The freezing comprises lowering the temperature of the fraction below 0° C. down to a cryopreservation temperature, for example in the range from -80° C. to -200° C. Freezing parameters for the freezing are for example the time profile of the temperature lowering, and the set cryopreservation temperature.

The pretreatment methods and freezing methods of the cryopreservation are carried out in a manner known per se; however, according to the invention, specific pretreatment methods and/or freezing methods are used in the cryopreservation of the cell clusters of each of the at least two fractions. For each fraction, different process parameters, in particular pretreatment and/or freezing parameters, are provided. For each fraction, process parameters are selected such that the cryopreservation of the cell clusters is optimized, in particular to maintain maximum vitality and/or to maintain maximum functionality.

The application of process parameters for the cryopreservation comprises adjusting previously-selected pretreatment parameters and freezing parameters. Optimal pretreatment parameters and freezing parameters can be determined using series of tests using cell clusters and/or can be determined from reference experiments from the specialist literature. The selection according to the invention of sample-specific process parameters for the cryopreservation advantageously makes it possible to improve the yield, efficiency and/or applicability of the cryopreservation for different cell types.

After the freezing down to the cryopreservation temperature, the frozen fractions are preferably provided for storage in a cryobank without interrupting the cold chain. Storage takes place in the cryobank at a storage temperature which may deviate from the cryopreservation temperature.

Advantageously, there are a plurality of available properties which can be used as the basis for the fractionation of the cell clusters. According to preferred embodiments of the invention, the fractionation of the cell clusters takes place on the basis of at least one of the properties including a size, a shape, a mass, an elasticity, a hydraulic conductivity, a cryoprotectant (CPA) permeability, a resistance to cryoprotectants, a chemical property and a cell composition of the cell clusters. The fractionation device is accordingly preferably adapted for fractionation on the basis of at least one of these properties. The above physical and chemical properties have advantageously proven particularly suitable for effective fractionation and for the selection of optimized process parameters for the cryopreservation.

The cell clusters can be fractionated on the basis of a plurality of properties, for example on the basis of the size and the CPA permeability. In the fractionation according to multiple properties, a multi-stage fractionation is preferably provided, in which, in a first stage, a first property is tested, for example the size of the cell clusters, and in at least one further stage, at least one further property is tested, for example CPA permeability. Size-dependent fractionation is particularly preferably provided. For size-dependent separation, numerous gentle separation methods are available. The efficiency of process parameters for cryopreservation may be particularly acutely dependent on the size of a cell cluster. The size of a cell cluster includes for example the cross-sectional dimension thereof, in particular the diameter thereof, or another characteristic geometrical measure of the cell cluster which has an effect on substance transport and/or the freezing process. Alternatively or additionally, fractionation into fractions with specific shapes of the respective cell clusters is particularly preferred. The shape of a cell cluster is the geometric form at least approximately assumed by the cell cluster in the surrounding medium, for example a spherical shape or an elongated cylindrical shape or an irregular shape.

In the cryopreservation, each fraction is subjected to a pretreatment which is characterized by fraction-specific selected pretreatment parameters. According to further preferred embodiments of the invention, the pretreatment methods for the fractions differ in terms of at least one of the pretreatment parameters including a duration, a temperature, a pressure, a medium composition, a gas supply composition, permeabilization conditions and a movement of the medium of the pretreatment. These pretreatment parameters have advantageously proven particularly well suited to a pretreatment of the cell clusters for effective cryopreservation with a high yield.

Advantageously, according to further variations of the invention, the pretreatment methods for the fractions may differ in terms of the time profile of at least one of the pretreatment parameters. The pretreatment methods may be characterized by different time dependencies of the pretreatment parameter. The time dependency advantageously achieves an additional degree of freedom in the optimization of the pretreatment.

According to further preferred variants of the invention, it is provided that the freezing methods for the fractions differ in terms of at least one of the freezing parameters including a duration, in particular a cooling rate, a temperature, a pressure, a medium composition, a gas supply composition, and a movement of the medium of the freezing. The freezing device is accordingly preferably adapted to apply the freezing method having at least one of said freezing parameters. This advantageously means that a plurality of parameters are available which can be used to optimize the cryopreservation for different fractions each having equal cell clusters.

If, according to a further preferred embodiment of the invention, the fractionation of the cell clusters comprises fluidic fractionation, in which the cell clusters are separated in a fluid environment, this gives rise to further advantages since the cell clusters can be kept in a liquid medium from their preparation, in particular their culturing, right up to the freezing, preventing any temporary transitions into a gaseous or vaporous environment.

According to an advantageous embodiment of the invention, the fractionation and cryopreservation are carried out in an automated manner. The cryopreservation apparatus is adapted for automated operation, in particular operation without intervention by an operator of the cryopreservation apparatus. Automation affords advantages in terms of preventing process errors, reproducibility and accuracy of the setting of process parameters, and the possibility of carrying out high-throughput fractionation by machine into separate fractions with downstream cryopreservation of the separate fractions at high speed and at high throughput.

According to a further modification of the invention, if the fractionation of the cell clusters comprises fractionation, in particular size-based fractionation, in a fluid flow, the cell clusters are arranged at different positions in a flow profile of the fluid flow under the effect of at least one of flow forces of the fluid flow, dielectrophoretic forces in the fluid flow, and sound waves in the fluid flow. The flow profile of the fluid flow comprises the location-dependent distribution of the flow rate in the cross section of the flow. By means of the different positions in the flow profile, this introduces separation of the cell clusters into different flow paths in the flow. The transfer into the different containers takes place by guiding the individual parts of the flow profile into the containers via separate partial flows, for example partial channels of a fluidic system, and/or in different directions.

Preferably, the fluidic system of the fractionation device is a fluidic microsystem, comprising channels and fluidic elements, for example branches and/or intersections, having characteristic cross-sectional dimensions of less than 2 mm. The fluid flow is particularly preferably a parallel, eddy-free flow, which advantageously improves the separation in the flow and makes it possible to guide the parts of the flow profile into the containers at a distance from the separation of the cell clusters into the various positions in the flow profile.

The fractionation by means of flow forces of the fluid flow is size-based fractionation by means of passive fluidics. Cell clusters become ordered at the various positions in the flow profile according to their sizes, and can thus be separated. Passive fluidics has the following advantages. It is a contactless method which puts only minimal strain on the cell clusters and does not require size sensors. The inhomogeneous mixture of cell clusters can be added in portions and separated by transit time differences (chromatographic principle, field flow fractionation). However, preference is given to continuous methods, for example pinched flow fractionation (PFF), which enable a more simple apparatus design and are more readily scalable. In PFF, for example, cell clusters of different sizes leave via an outlet, in particular a nozzle portion, at different angles.

Fractionation by means of dielectrophoretic forces in the fluid flow is a size-based fractionation or a fractionation according to an electrical property of the cell clusters using active fluidics, for example separation according to dielectric properties of the cell clusters, for example polarizability or surface charge. For this separation method, the fractionation device is preferably provided with an electrode device which is adapted to apply dielectrophoretic forces in a fluid flow. The electrode device comprises for example an electrode which, when an AC voltage is applied thereto, generates a dielectrophoretic field barrier which forms a deflection angle (not equal to 0°) with the flow direction in the fluidic system. The strength of the dielectrophoretic field barrier which acts on a cell cluster is dependent on the size of the cell cluster. Dielectrophoretic forces acting on the cell clusters at right angles to the direction of flow are superimposed with flow forces in the flow. Depending on their size and/or dielectric properties and on the flow forces, cell clusters can pass the electrodes at different positions and can be positioned accordingly in the flow profile. Advantageously, this also gives a contactless method which does not require any upstream sensors. However, the apparatus expense is higher than for passive fluidics.

Alternatively, the fractionation by means of dielectrophoretic forces can be combined with sensors. A sensor device can be arranged upstream of the electrode device, which sensor device is adapted for detecting at least one property of the cell clusters. The electrode device is controlled on the basis of an output signal from the sensor device such that the individual cell clusters are guided to the various positions in the flow profile on the basis of the detected property.

Fractionation by means of sound waves is performed in the fluid flow; another variant of size-based fractionation thus is provided by active fluidics. Acoustic fields (standing and/or travelling sound waves) of suitable frequency, and the ability of inert bodies to accumulate at the field minima of sound waves, are used for fractionation. In this case, too, this gives a contactless method which does not require any upstream sensors. The size range over which acoustic fractionation can be used is advantageously greater than that for dielectrophoretic fractionation. For this separation method, the fractionation device is preferably provided with a sound source device which is adapted to generate sound waves in the fluid flow.

According to a further preferred embodiment of the invention, the at least one property of the cell clusters and/or at least one state variable of the at least two fractions are detected by means of sensing. Accordingly, the cryopreservation apparatus is preferably provided with a sensor device, which is adapted for detecting the at least one property of the cell clusters and/or at least one state variable of the at least two fractions. The detection by means of sensing of the at least one property of the cell clusters particularly preferably takes place directly before the fractionation, and the detection by means of sensing of the at least one state variable of the fractions particularly preferably takes place directly before the cryopreservation. The sensing of the at least one property of the cell clusters before the fractionation advantageously increases the group of properties of the cell clusters which can be used as a basis for the fractionation. The sensing of the at least one state variable of the fractions before the cryopreservation affords the advantage of further optimizing the process parameters for the cryopreservation on the basis of the state of the fraction. State variables of the fraction are for example the density or the size of the cell clusters.

A further particularly important advantage of the invention for the further use of the cell clusters after the cryopreservation is that the vitality-maintaining thawing can also take place in a fraction-specific manner. According to an advantageous variant of the invention, the thawing of the cell clusters of the at least two fractions takes place such that specific thawing methods are used for each fraction. Thawing parameters are, like the process parameters for the cryopreservation, optimized separately for the individually fractionated fractions, enabling an increase in the vitality rate of the thawed cell clusters.

In general, a method for the vitality-maintaining thawing of at least two fractions, which were obtained and frozen by fractionation of cell clusters on the basis of at least one property of the cell clusters, with a specific thawing method being used for each fraction, and a thawing device which is configured to carry out the method, can be considered to be further independent subjects of the present invention.

The features disclosed in conjunction with the method for cryopreserving a plurality of cell clusters of biological cells, and the embodiments thereof, are likewise preferred features of the cryopreservation apparatus or the embodiments thereof. The above-mentioned aspects and preferred features according to the invention, in particular with regard to the method, therefore also apply to the cryopreservation apparatus and the components thereof.

Further details and advantages of the invention will be described below with reference to the appended drawings. The drawings show, schematically:

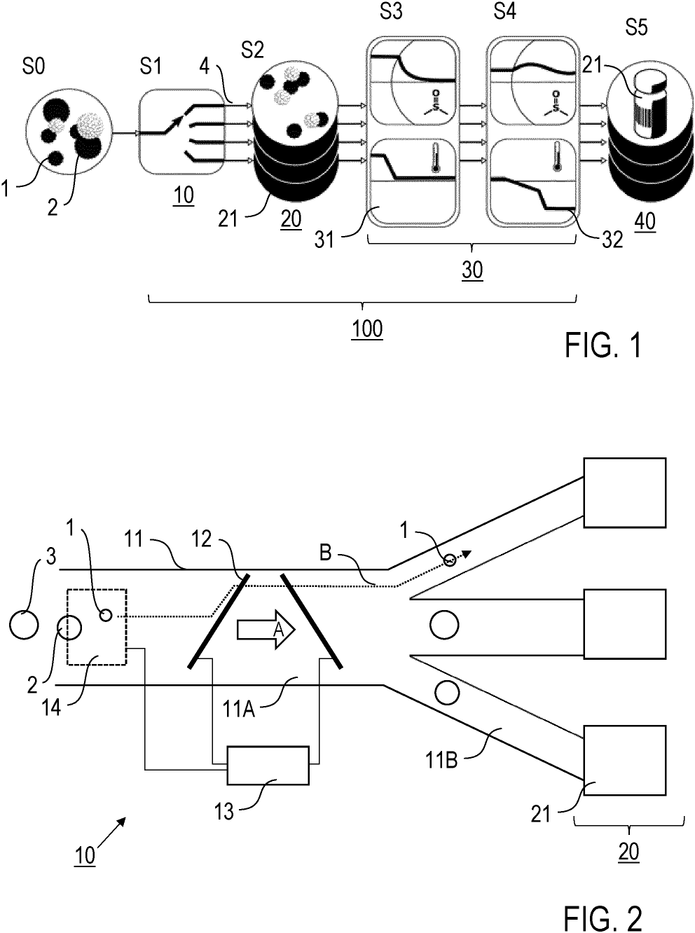

FIG. 1: a sequence of the method for cryopreserving a plurality of cell clusters and components of a cryopreservation apparatus having features according to preferred embodiments of the invention; and

FIG. 2: a fractionation device which is adapted for the dielectrophoretic separation of cell clusters, according to an embodiment of the invention.

Features of preferred embodiments of the invention are described below by means of exemplary reference to the use of size-based fractionation of cell clusters. It is emphasized that, in practice, implementation of the invention is not limited to size-based fractionation but rather is also possible, alternatively or additionally, with fractionation based on another property of the cell clusters, as is described below with further examples. Details of the cell clusters and their preparation, and process parameters for cryopreservation and/or thawing parameters used in concrete examples are selected as is known per se from the cryopreservation of biological materials.

FIG. 1 shows steps S1 to S4 of the method for cryopreserving a plurality of cell clusters 1, 2, and the cryopreservation apparatus 100 used to this end, having a fractionation device 10, a container device 20 and a freezing device 30 according to preferred embodiments of the invention. FIG. 1 additionally shows a step S0 of preparing the cell clusters 1, 2 and a step S5 of storing the frozen cell clusters in a cryobank 40. The example shown in FIG. 1 is used to carry out for example an automated, fluidic size-based fractionation of the cell clusters 1, 2.

With step S0, an inhomogeneous sample is prepared, for example a mixture of cell clusters 1, 2 of different sizes and/or a mixture of cell clusters 1, 2 having different sensitivities to CPA. The cell clusters 1, 2 comprise for example organoids which were formed in a known way by culturing in a nutrient medium and using differentiation factors from adult stem cells and which for example have cross-sectional dimensions in the range from 10 µm to 10 mm or greater. The cell clusters 1, 2 are prepared for example in a culture vessel.

With step S1, the cell clusters 1, 2 are separated into individual fractions 4 (fractions), each of which contains cell clusters of specific sizes, using the schematically depicted fractionation device 10. The fractionation device 10 is for example constructed as described below with reference to FIG. 2, and it is preferably operated in an automated manner. The fractions 4 are transferred into container 21 of the container device 20 in step S2. The containers 21 preferably comprise plastic tubes with a lid, in particular what are referred to as PP tubes, as are used in the subsequent cryopreservation in steps S3 and S4 (see illustration in step S5). Alternatively, the containers may comprise other receptacles, for example pouches or microtiter plates. According to a further alternative, the containers 21 may be part of the incubation unit 31 of the freezing device 30. The individual fractions 4 are collected in the containers 21 provided for storage, until a predetermined loading quantity, in particularly concentration (mass of cell clusters per unit volume of surrounding medium), is reached.

The freezing device 30 comprises an incubation unit 31 and a cooling unit 32. In the freezing device 30, the individual fractions 4 are subjected to a pretreatment and freezing protocol which is adapted to the size in question.

Pretreatment of the fractions takes place in the incubation unit 31. This means that a complete and individual incubation program is run for each fraction 4. In the process, at least a CPA (in particular a cryoprotectant) is added, with which the cell clusters are intended to be loaded. As the size of the cell clusters increases, for example increasing concentrations of CPA and/or increasing incubation times are used. Suitable cryoprotectants and the concentrations thereof can be determined using tests.

The incubation can furthermore comprise a predetermined temperature conditioning T(t), supply of gas and/or perfusion with predetermined cryoprotectant (CPA) concentration profiles C(t, CPA1, CPA2, ...). Alternatively or additionally, temporarily membrane-permeable and/or even toxic CPA can be supplied, if the cell clusters tolerate same. Furthermore, ice nucleation (for reducing and controlling supercooling), medium circulation (for homogenizing T and C), and/or permeabilization of the cell clusters of at least one fraction (for loading with non-membrane-permeable CPA) may be part of the pretreatment method. Permeabilization can take place for example chemically (for example by means of DMSO), using sound waves (sonoporation), using electric fields (electroporation), by means of liposomal substances and/or by thermomodulation via membrane phase conversion. Furthermore, the pretreatment in the incubation unit 31 comprises pre-cooling of the fractions 4 to a temperature above the freezing point of the fractions 4.

The incubation unit 31 preferably has individual receptacles for the containers 21, for example individual cavities, or provides the containers by reservoirs, preferably for fractions of the same volume. The incubation unit 31 includes a pump device for supplying CPA (sequential addition and/or concentration increase) and/or for discharging media from the containers. Furthermore, the incubation unit 31 is preferably provided with a drive, for example a stirring mechanism, for moving the media during the pretreatment in each container. Alternatively or additionally, a pre-cooling unit can be provided, which is adapted for supercooling fractions. Supercooling can induce membrane changes in the cells of the cell clusters, as a result of which the pretreatment, for example the uptake of CPAs, can be influenced. Alternatively or additionally, a sound source can furthermore be provided, by means of which the cell clusters of the fractions can be subjected to an ultrasonic treatment. The ultrasonic treatment can induce further membrane changes in the cells of the cell clusters, in particular permeabilization.

Further pretreatment parameters of the size-dependent incubation include for example concentrations of the individual CPAs, durations of action of the individual CPAs, concentration profiles over time of individual CPAs, adapted temperature profiles (> 0° C.), continual changes in the medium composition, for example by means of mixing devices in conjunction with the incubation unit 31 and a CPA reservoir, and/or a change in the surrounding medium (perfusion).

Subsequently, the fractions 4 are frozen in the cooling unit 32 (step S4). Depending on the properties of the cell clusters of the fractions 4, for example the size or other properties, such as the hydraulic conductivity of the individual components of the cell clusters, the proportion of membrane-permeable and osmotically-active additives in the medium and/or the supercooling, the individual fractions 4 are frozen in a controlled manner at different cooling rates and/or with different cooling profiles. For example, a cooling rate which is equal to or less than -1 K/min is used. The cooling takes place down to a cryopreservation temperature of for example -80° C. or lower, for example -140° C. or lower.

Temperature profiles for the freezing of individual fractions can be selected for example in order to set an adaptation to an equilibration rate, a controlled nucleation for reducing supercooling, and/or a homogeneous cooling rate, via the fraction volume (optionally with circulation of the medium and/or with use of a form-fit of the fraction container in the heat exchanger of the cooling unit 32).

For each fraction, the cooling unit 32 comprises a cooling chamber having at least one cooling element and a heat exchanger. The cooling element is for example a Peltier element, a Stirling cooler or a coolant flow cooler, operating for example with liquid nitrogen or isopentane. The cooling element is adapted for setting a defined cooling rate. The heat exchanger comprises for example a receptacle for the container of each respective fraction, preferably with a form-fit between the container and the receptacle. If the containers 21 are part of the incubation unit 31 of the freezing device 30, transfer into cryocontainers, for example said PP tubes, takes place before the freezing. The cooling unit 32 can optionally be provided with a nucleation apparatus, for example a cold needle, by means of which controlled nucleation can be induced in the container.

Finally, the containers are closed and the frozen fractions are stored in the cryobank 40 at cryogenic temperatures (for example -140° C.) (step S5). The transfer to the cryobank 40 takes place without interrupting the cold chain, for example using a cooled sluice or by directly coupling the freezing device 30 to the cryobank 40.

For thawing, the process shown in FIG. 1 is reversed using a thawing device (not shown). During thawing, like the size-adapted process for freezing, the individual fractions are also treated separately in different incubation units during the thawing and/or in a first recovery phase until the auxiliary agents are washed away. For example, a size-dependent thawing rate, a size-dependent incubation in a hyperosmolar thawing medium and/or a size-dependent washing out of the thawed fractions is provided. As a result, for example, the metabolism of relatively large cell clusters can be slowed by a cooled environment in order to ensure sufficient dilution of toxic, membrane-permeable CPAs, while this process can take place more quickly in the case of small cell clusters.

After the thawing, a portionation step can be provided in which the thawed fractions are subjected to a vitality test and, when vitality is detected, are transferred to a predetermined usage-dependent container format, for example microtiter plates or suspension bioreactors. In this case, the size-based fractionation can be maintained or dispensed with.

The thawing and/or portionation can be carried out for example using a fluidics device, in particular a fluidic microsystem.

FIG. 2 shows by way of example a fractionation device 10 in the form of a fluidics device 11, in particular a fluidic microsystem, having a main channel 11A and branching channels 11B, through which a suspension of a liquid surrounding medium having cell clusters 1, 2, 3 of different sizes flows, in the direction of arrow A. An electrode device 12 and a sensor device 14, which are connected to a control device 13, are located in the main channel 11A. The main channel 11A branches into the branching channels 11B, each of which are connected to a container 21 of the container device 20.

The electrode device 12 comprises two strip-like electrodes or electrode pairs, for example at the bottom and/or on a cover plate of the main channel 11A. When AC voltages from the control device 13 are applied to the electrodes, the electrode device 12 can produce a field barrier at right angles to the flow A. The field barrier can be generated temporarily to match a cell cluster arriving with the flow. By interaction of the field barrier with the flow forces in flow A, cell clusters can be guided onto a predetermined flow path which leads to one of the branching channels 11B (see for example the dotted flow path B of cell cluster 1).

The sensor device 14 is for example an optical sensor, in particular a camera connected to an image processing device. The cell clusters 1, 2, 3 and their respective sizes can be detected by means of the sensor device 14. Information regarding the positions and sizes of the cell clusters 1, 2, 3 is delivered to the control device 13. The control device 13 assigns the cell clusters 1, 2, 3 to three predetermined sizes of the desired fractions and controls the electrode device 12 such that the cell clusters 1, 2, 3 are each guided based on their sizes into one of the branching channels 11B and via same into one of the containers 21.

As an alternative to the embodiment in FIG. 2, the electrode device 12 can comprise a dielectrophoretic field cage and the sensor device 14 can be adapted to detect a cell cluster in the field cage. The cell clusters are detected one after the other in the field cage by means of sensors, assigned to one of a plurality of fractions on the basis of their properties, and guided into the fraction in question by releasing the field cage and optional further dielectrophoretic deflection.

Alternatively or additionally to the size-based fractionation using dielectrophoretic forces, at least one of the following separation methods may be provided for fractionation. Passive separation methods can include for example fractionation in the flow profile (for example PFF), density-based fractionation (for example sedimentation), and geometric fractionation (for example using screens). Active separation methods can include for example acoustic separation (for example with standing ultrasound waves) or optical separation (for example using optical tweezers).

Alternatively to optical sensors, for example an impedance measurement at the cell clusters may be provided (for example as in a “Coulter Counter” device), and the fractionation can be carried out on the basis of the result of the impedance measurement.

The size-based fractionation described with reference to FIGS. 1 and 2 can be supplemented by fractionation in relation to other properties, or replaced thereby. For example, in a fluidics device of a fractionation device, for example in a field cage of the fluidics device, a test of the permeability of the cells of cell clusters to cryoprotectants and/or a resistance to cryoprotectants can be carried out. In dependency on the test result, different fractions can be formed, which are subsequently subjected to cryopreservation using different process parameters. For example, cell clusters with low CPA permeability of the cells are treated using a longer CPA incubation duration than cell clusters with increased CPA permeability of the cells.

The features of the invention disclosed in the preceding description and in the drawings and claims may be significant, both individually and in combination or in subcombinations, for implementing the invention in the various embodiments thereof.

1. A method for cryopreserving a plurality of cell clusters of biological cells, comprising the steps of:
fractionating the cell clusters into at least two fractions in dependency on at least one property of the cell clusters,
collecting the fractions in different containers, and
cryopreserving the cell clusters of the at least two fractions, wherein at least one of specific pretreatment methods and specific freezing methods are used for each fraction.

2. The method according to claim 1, wherein
the fractionating of the cell clusters takes place on a basis of at least one property selected from the group consisting of a size, a shape, a mass, an elasticity, a hydraulic conductivity, a cryoprotectant permeability, a resistance to cryoprotectants, a chemical constitution and a cell composition of the cell clusters.

3. The method according to claim 1, wherein
the pretreatment methods for the fractions differ in terms of at least one pretreatment parameter selected from the group consisting of a duration, a temperature, a pressure, a medium composition, a gas supply composition, permeabilization conditions and a movement of a medium of the pretreatment.

4. The method according to claim 3, wherein
the pretreatment methods for the fractions differ in terms of a time profile of at least one of the pretreatment parameters.

5. The method according to claim 1, wherein
the freezing methods for the fractions differ in terms of at least one of the freezing parameters including a duration, a temperature, a pressure, a medium composition, a gas supply composition, and a movement of the medium of the freezing.

6. The method according to claim 1, comprising at least one of the features:
the fractionating of the cell clusters comprises fluidic fractionation, in which the cell clusters are separated in a fluid environment,
the fractions are collected in the containers in which the cryopreserving subsequently takes place to provide frozen fractions,
the frozen fractions are provided for storage in a cryobank without interrupting a cold chain, and
the fractionating and cryopreserving are carried out in an automated manner.

7. The method according to claim 1, wherein
the fractionating of the cell clusters comprises fractionation in a fluid flow, wherein the cell clusters are arranged at different positions in a flow profile of the fluid flow under an effect of at least one of flow forces of the fluid flow, dielectrophoretic forces in the fluid flow, and sound waves in the fluid flow.

8. The method according to claim 1, wherein
at least one of the at least one property of the cell clusters and at least one state variable of the at least two fractions are detected by sensing.

9. The mMethod according to claim 1, further comprising the additional step of
thawing the cell clusters in the at least two fractions, wherein specific thawing methods are used for each fraction.

10. A cryopreservation apparatus, which is configured for cryopreserving a plurality of cell clusters of biological cells, comprising:
a fractionation device, which is configured for fractionating the cell clusters into at least two fractions in dependency on at least one property of the cell clusters,
a container device having at least two different containers, each of which is arranged for collecting one of the fractions, and
a freezing device, which is configured for cryopreserving the cell clusters in the at least two fractions, wherein the freezing device is configured for using at least one of specific pretreatment methods and specific freezing methods for each of the fractions.

11. The cryopreservation apparatus according to claim 10, wherein
the fractionation device is configured for fractionating the cell clusters on a basis of at least one property selected from the group consisting of a size, a shape, a mass, an elasticity, a hydraulic conductivity, a cryoprotectant permeability, a resistance to cryoprotectants, a chemical constitution and a cell composition of the cell clusters.

12. The cryopreservation apparatus according to claim 10, wherein
the freezing device is configured to apply the pretreatment methods which differ in terms of at least one of: (a) a pretreatment parameters; and (b) a time profile of said pretreatment parameter, wherein said pretreatment parameter is selected from the group consisting of a duration, a temperature, a pressure, a medium composition, a gas supply composition, permeabilization conditions and a movement of a medium during the pretreatment.

13. The cryopreservation apparatus according to claim 10, wherein
the freezing device is configured to apply the freezing methods which differ in terms of at least one freezing parameter selected from the group consisting of a duration, a temperature, a pressure, a medium composition, a gas supply composition, and a movement of a medium during the freezing.

14. The cryopreservation apparatus according to claim 10, comprising at least one of the features
the fractionation device comprises a fluidics device which is configured for separating the cell clusters in a fluid environment,
the at least two containers are part of the freezing device, and
the cryopreservation apparatus is configured for automated operation.

15. The cryopreservation apparatus according to claim 10, wherein
the fractionation device comprises at least one of an electrode device which is configured to apply dielectrophoretic forces in a fluid flow, and a sound source device which is configured to generate sound waves in the fluid flow.

16. The cryopreservation apparatus according to claim 10, comprising
a sensor device which is configured for detecting at least one of the at least one property of the cell clusters and at least one state variable of the at least two fractions.

17. The cryopreservation apparatus according to claim 14, comprising the fluidics device, which is configured for separating the cell clusters in a fluid flow.
